(19) United States
(12) Patent Application Publication
(10) Pub. No.: US 2005/0201945 A1
(43) Pub. Date: Sep. 15, 2005

(54) Medicament dispenser

(76) Inventors: Cecile Isabelle Bonvoisin, Paris (FR); Ignatius Loy Britto, Evreux (FR); Ralf Greger, Oudenaarde (BE); Christophe Laroche, Evreux (FR); Verna Charlene Lo, Durham County, NC (US); Johan Palmers, Amougies (BE); Isabelle Denise Peyron, Evreux (FR); Anthony Vanlandeghem, Oudenaarde (BE); Hirotsugu Yasuda, Columbia, MO (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/493,327

(22) PCT Filed: Oct. 23, 2002
(86) PCT No.: PCT/GB02/04794

§ 371 Date: Mar. 29, 2005

(30) Foreign Application Priority Data
Oct. 23, 2001 (GB) 01253806

(51) Int. Cl. A61M 11/00; A61L 9/04

(52) U.S. Cl. 424/45; 128/200.23

(57) ABSTRACT

A dispenser for dispensing a medicament that includes a canister for housing the medicament and a fluid propellant therefor and a drug-dispensing valve wherein one or more of the internal surfaces of the canister and/or valve includes a fluorinated coating prepared from plasma polymerisation of one or more fluorinated monomers selected from the group consisting of CH₂FCF₃ and C₃F₆ is disclosed.

### RELATED APPLICATION

The present application claims priority from UK patent application No. 0125380.6 filed on 23 Oct. 2001, the entire content of which is hereby incorporated herein by reference.

### FIELD OF THE INVENTION

The present invention relates to a dispenser for a metered dose inhaler. More especially, the invention relates to a dispenser for a metered dose inhaler for consistently dispensing a prescribed dose of medicament.

### BACKGROUND OF THE INVENTION

Drugs for treating respiratory and nasal disorders are frequently administered in aerosol formulations through the mouth or nose. One widely used method for dispensing such aerosol drug formulations involves formulating the drug as a suspension or a solution in a liquefied gas propellant. The suspension/solution is stored in a sealed canister capable of withstanding the pressure required to maintain the propellant as a liquid. The suspension/solution is dispersed by activation of a dose-metering valve affixed to the canister.

A metering valve generally comprises a metering chamber, which is of a set volume and is designed to administer per actuation an accurate predetermined dose of medicament. As the suspension/solution is forced from the metering chamber through the valve stem by the high vapour pressure of the liquid propellant, the propellant rapidly vaporises leaving a fast moving cloud of very fine particles of the drug formulation. This cloud of particles is directed into the nose or mouth of the patient by a channelling device such as a cylinder or open-ended cone.

Concurrently with the activation of the aerosol dose-metering valve, the patient inhales the drug particles into the lungs or nasal cavity. Systems of dispensing drugs in this way are known as “metered dose inhalers” (MDIs). See Peter Byron, Respiratory Drug Delivery, CRC Press. Boca Raton, Fla. (1990) for a general background on this form of therapy.

Patients often rely on medication delivered by MDIs for rapid treatment of respiratory disorders, which are debilitating and in some cases even life threatening. Therefore, it is essential that the prescribed dose of aerosol medication delivered to the patient consistently meets the specifications claimed by the manufacturer and meets the requirements of regulatory authorities. That is, every dose in the can must be delivered within the same close tolerances.

A problem which can exist with drug delivery devices such as MDIs is deposition of medicament, or the solid component from a suspension of a particulate product in a liquid propellant, onto the internal surfaces of the device which occurs after a number of operation cycles and/or storage. A reduction in the efficacy of the device may occur. Deposition of the product also reduces the amount of active drug available to be dispensed to the patient and markedly reduces the uniformity of the dose dispensed during the lifetime of the device.

Drug deposition and adherence and dose uniformity may be greater with suspension formulations comprising hydrofluoroalkane propellants, for example, 1,1,1,2-tetrafluoroethane (HFA134a) and 1,1,1,2,3,3,3-n-heptafluoropropane (HFA227), which have been developed as ozone friendly replacements of chlorofluorocarbons such as P11, P114 and P12.

Some conventional devices rely on the dispenser being shaken, to agitate the liquid propellant and product mixture therein, in an attempt to re-suspend at least a portion of the deposited medicament. While in some cases this remedy can be effective within the body of the drug container itself, it may not be effective for particles deposited on the inner surface(s) of other MDI components, such as the metering valve.

Canadian patent application 2130867 describes a metered dose inhaler containing an aerosol formulation in which the internal walls of the metal canister are coated with a cross-linked plastics coating. Polytetrafluoroethylene (PTFE) and perfluoroethylenepropylene (FEP) are specifically mentioned as suitable coating materials

UK patent application GB-A-2,328,932 discloses the use of a liner of a material such as fluoropolymer, ceramic or glass to line a portion of the wall of the metering chamber in a metering valve of an MDI. Although this alleviates the problem of deposition in these types of dispensers, it does require the re-design or modification of mouldings and mould tools for producing the valve members to allow for insertion of the liner.

### SUMMARY OF THE INVENTION

It is therefore an aim of the present invention to provide a highly fluorinated, reproducible coating which prevents or inhibits adhesion of drug particles to the internal surfaces of the canister and/or valve components of a medicament dispenser, for example an MDI.

It is a further aim of the present invention to provide a coating with reduces moisture ingress into a medicament formulation, for example a pharmaceutical aerosol formulation, reduces drug absorption into the internal surface, especially when of rubber, and reduces extractables leached out from the internal surface, especially when of plastics and rubber components.

### DETAILED DESCRIPTION OF THE INVENTION

Accordingly, in a first aspect, the invention provides a dispenser for dispensing a medicament comprising a canister for housing the medicament and a fluid propellant therefor and a drug-dispensing valve wherein one or more of the internal surfaces of the canister and/or valve comprises a fluorinated coating prepared from plasma polymerisation of one or more fluorinated monomers selected from the group consisting of CH₂FCF₃ and C₃F_{6.}

In a first embodiment, the coating is prepared from plasma polymerisation of a CH₂FCF₃ monomer.

In a second embodiment, the coating is prepared from plasma polymerisation of a C₃F₆ monomer.

Suitably, the fluorinated coating has a fluorine/carbon atomic ratio of greater than 10% about 1.0 and preferably greater than about 1.2, when measured by Electronic Spectroscopy for Chemical Analysis (ESCA), also referred to as X-ray photo spectroscopy (XPS).

Suitably, the fluorinated coating comprises greater than about 10% CF₂ units and greater than about 10% CF₂CF units, the CF₂ and CF₂CF units being present either as part of a Teflon moiety or as a separate moiety. The percentage of CF₂ and CF₂CF units may be measured using ESCA.

Suitably, the surface energy of the coating gives a contact angle of greater than about 80 degrees, preferably greater than about 90 degrees. The term “contact angle” is the angle between a liquid water droplet and the coated surface of the canister/valve at the liquid/solid interface as measured in ambient conditions, i.e. at a temperature of 20° C. (±5° C.) and a relative humidity of 50% (±20%). The contact angle may be measured on a coating deposited on a flat polybutylene terephthalate (PBT) substrate surface in accordance with the invention.

The thickness of the fluorinated coating is in the range of about 1 to about 200 nm, suitably about 10 to 100 nm, and preferably about 20 to 80 nm.

In one embodiment, one or more internal surfaces of the canister comprise the fluorinated coating of the invention. In addition, or alternatively, one or more internal surfaces of the valve may comprise the fluorinated coating of the invention.

Any parts of the canister or valve which contact the pharmaceutical aerosol suspension may be coated with the fluorinated coating of the invention. The fluorinated coating reduces or eliminates the tendency for medicament particles to adhere to such component surfaces. Where the valve part is a movable part (e.g. the valve stem) the coating also reduces the friction between that part and an adjacent part of the valve (e.g. the stem seal).

As known by a person skilled in the art, the drug-dispensing valve suitably comprises a number of components or parts. All of these may, independently of the other components, be coated with a fluorinated coating as hereinbefore defined. Component parts of the valve which may be coated include, but are not limited to, the metering chamber, valve stem, the upper and lower stem seals, neck gasket, spring, body, and the ring.

In one aspect herein, the valve stem is provided with the coating of the invention to reduce its frictional contact properties, and the need for any further stem lubricant such as silicone oil is reduced or eliminated. Reducing frictional contact can be particularly advantageous where the valve is employed in a dispenser for both suspension and solution medicament formulations.

In a further aspect, one or more internal surfaces of the metering chamber are provided with a fluorinated coating according to the present invention.

In a still further aspect, one or more component parts selected from the group consisting of the upper and lower stem seals, neck gasket, spring, body, and ring are provided with a fluorinated coating according to the present invention.

In another aspect, the invention provides a drug-dispensing valve for use in a dispenser for dispensing a medicament in a fluid propellant, wherein one or more of the internal surfaces of said valve comprise a fluorinated coating prepared from plasma polymerisation of a fluorinated monomer selected from the group consisting of CH₂FCF₃ and C₃F₆.

In a further aspect, the invention provides a canister for housing the medicament in a fluid propellant, wherein one or more of the internal surfaces of said canister comprise a fluorinated coating prepared from plasma polymerisation of a fluorinated monomer selected from the group consisting of CH₂FCF₃ and C₃F₆.

The dispenser and/or drug-dispensing valve and/or canister as hereinbefore defined may be incorporated as part of a “metered dose inhaler” (“MDI” for short) for dispensing a medicament in a fluid propellant under pressure. The term “MDI” means a unit comprising a canister, a ferrule covering the mouth of the canister, a drug metering valve situated in the ferrule, a metering chamber and a suitable channelling device into which the canister is fitted. The relation of the parts of a typical MDI is illustrated in U.S. Pat. No. 5,261,538, the content of which is hereby incorporated herein by reference. In another aspect, the invention provides a metered dose inhaler for dispensing a medicament in a fluid propellant, comprising a dispenser and/or a drug-dispensing valve and/or a canister as defined above and a medicament channelling device, such as an actuator.

Optionally, moisture-absorbing means is further comprised within the dispenser and/or drug-dispensing valve and/or canister and/or metered dose inhaler of the invention as a component thereof. Examples of moisture absorbing means suitable for use with the present invention are disclosed in co-pending UK Patent Application 0116891.3, the content of which is hereby incorporated herein by reference.

The coating applied to one or more internal surfaces of the canister and/or valve is prepared from a plasma generated substantially from a fluorinated monomer selected from the group consisting of CH₂FCF₃ and C₃F₆. Alternatively, the fluorinated monomer selected from the group consisting of CH₂FCF₃ and C₃F₆ may be co-polymerised with one or more additional non-fluorinated monomers. Suitable copolymers comprise from 0.5 to 99.5% by weight, preferably from 0.7 to 85% by weight, of fluorinated monomer. In general the preference is to use a non-fluorinated monomer that forms the basic building block (monomer) of the substrate polymer or elastomer to be coated. For example, if polybutylene terephthalate (PBT) is the is substrate to be coated, the monomer used in producing PBT, dimethyl terephthalate, can be used in conjunction with the fluorinated monomer. Similarly, if the substrate is acetal, then CH₂O can be used. In general, irrespective of the substrate material, when fluorinated coatings are produced using a plasma process, it is desirable to use basic hydrocarbon monomers, including, but not limited to, CH₄, C₂H₆, C₂H₄, N₂, O₂, H₂, C₃COO(C₆H₆)COOCH₃, HO(CH₂)₂OH, C₃H₃N and C₄H₆ in conjunction with the fluorinated monomer.

The ratio of the gas flow rate of the fluorinated monomer to the non-fluorinated monomer can be continuously varied during the course of the plasma coating process. In general, in order to obtain superior adhesion, this ratio can be low or the monomer gas can be rich in the non-fluorinated species at the start of the process. This ratio can be continuously increased and towards the end of the process it is preferable to use only the fluorinated monomer in order to obtain a fluorine rich surface in the top layers of the coating.

The fluorinated coating of the invention is prepared using a plasma polymerisation process, suitably a RF plasma polymerisation process operating at a frequency of 2 MHz to 200 MHz; suitably 13.56 MHz, 27.12 MHz and 40.68 MHz; and preferably 13.56 MHz. The coating process typically occurs under vacuum. The components to be coated are placed inside a rotating chamber, the chamber subsequently being evacuated. The fluorinated monomer (and optionally additional monomeric material) is introduced into the chamber, suitably at ambient temperature, and at a controlled and predetermined flow rate. The monomer gas(es) is ignited and dissociates into plasma within the chamber. The energy in the chamber is maintained for a given time at a chosen power setting. During plasma polymerisation electrode temperatures can typically increase from about 20° C. to about 100° C. A cooling system of the electrode is used to minimise the temperature increase. At the end of the treatment the plasma is extinguished, the chamber flushed with air or argon and the coated products retrieved. During the polymerisation process, a thin layer of plasma polymer will be bonded to the canister and/or valve component. The polymerisation process time may only be minutes, for instance 30 minutes or less, or as long as several hours, depending on the operating conditions etc., as will be understood by the skilled reader in the art.

Accordingly, a further aspect of the invention provides a process for coating one or more of the internal surfaces of the canister and/or valve component with a fluorinated coating, said process comprising the steps of (i) placing the canister and/or valve component to be coated in a chamber, (ii) evacuating the chamber, (iii) feeding the fluorinated monomer selected from the group consisting of monomer CH₂FCF₃ and C₃F₆ into the chamber, (iv) applying sufficient power to generate a plasma, (v) igniting the plasma, (vi) extinguishing any unreacted plasma, and (vii) flushing the chamber.

One or more additional non-fluorinated monomers may also be fed into the chamber. Suitably, the ratio of fluorinated to non-fluorinated gas flow rate is continuously varied during the process. More suitably, the ratio of fluorinated to non-fluorinated gas flow rate is increased during the process. Preferably, the monomer gas is pure non-fluorinated monomer at the start of the process and pure fluorinated monomer at the end of the process.

The effectiveness of the fluorinated coating of the invention may depend on the operating conditions of the plasma reactor. The operating parameters, which can be varied, include: power (W), gas pressure (mTorr), gas flow (cc/min), tumbler speed (rpm), temperature (° C.) and the number of components in the chamber.

Suitably, the reactor operates at a power of between 50 W and 450 W, suitably 75 W and 300 W and preferably about 200 W.

Suitably, the reactor operates at a gas pressure of less than or equal to about 70 mTorr.

Suitably, the reactor operates at a gas flow of between 50 cc/min and 200 cc/min, suitably between 75 cc/min and 100 cc/min.

Suitably, the reactor operates at a tumbler speed of between 1 and 15 rpm, suitably at about 3 rpm or 8 rpm.

Suitably, the temperature of the electrode increases from 20° C. to 100° C.

The positioning of the components within the reactor may affect the effectiveness of the coating. The components to be coated should be positioned within the primary plasma in the reactor (inside the glow of the plasma). In order to obtain a uniform coating on all the components, the components should be evenly distributed in the reactor and then rotated.

Suitably, to improve adhesion of the fluorinated coating to the internal surfaces, the surfaces to be coated may be subjected to a pre-treatment procedure to remove any surface contamination and/or to activate the surface. Accordingly, a further aspect of the invention provides a dispenser for dispensing a medicament in a fluid propellant, the dispenser comprising a canister for housing the medicament and a drug dispensing valve, wherein one or more of the internal surfaces of the canister and/or valve are subjected to a pre-treatment step to remove surface contamination and/or to activate the surface prior to providing a fluorinated coating as hereinbefore described. The pre-treatment step may be carried out by for example plasma treatment of the components with an etching gas such as oxygen or a neutral gas such as argon. Preferably, the gas is argon to avoid damage to the substrate. In the process, radicals react with the plastic or metal substrate; for example the component is exposed to a low pressure argon plasma environment generating polar groups on the component's surface. Such polar groups are more conducive to bonding with the fluorine-containing plasma coating to be applied.

The pre-treatment step, for example with argon, could be carried out under a range of conditions and duration. However, the following conditions provide a satisfactory pre-treatment for a PBT substrate: run time 5 minutes; power 300 W; gas pressure 80 mTorr; gas flow 150 cc/min; tumbler speed 3 rpm or 8 rpm. It should be noted, however, that the invention is not limited to these conditions and that any set of conditions used for a pre-treatment step is within the scope of the invention. The pre-treatment process is dependent on the material to be treated.

The metered dose inhalers may be prepared by methods known in the art, for example as disclosed in Byron supra and U.S. Pat. No. 5,345,980, the content of each of which is hereby incorporated herein by reference.

Suitably, the entire valve or one or more of the valve components are made of a non-metal material. Suitable non-metals for use in the valve include pharmacologically resilient polymers such as acetal, polyamide (e.g. Nylon®), polycarbonate, polyester (e.g. polybutylene terephthalate (PBT)), fluorocarbon polymer (e.g. Teflon®) or a combination of these materials. Additionally, seals and “O” rings of various materials (e.g., nitrile rubbers, polyurethane, acetyl resin, fluorocarbon polymers), or other elastomeric materials, for example EPDM, and thermoplastic elastomer or chloroprene, are employed in and around the valve. Alternatively, the valve is made of metal, for example stainless steel, aluminium, copper, tin plate and any alloys thereof.

The valve can have any suitable configuration. Metal and non-metal parts can be combined to optimise the performance of the valve.

Conventionally, the canisters and caps for use in MDIs are made of aluminium or an alloy of aluminium although other metals not affected by the drug formulation, such as stainless steel, an alloy of copper, or tin plate, may be used. An MDI canister may also be fabricated from glass or plastics. Preferably, however, the MDI canisters and caps employed in the present invention are made of aluminium or an alloy thereof.

The canister, when in use, is a pressurised container comprising a vial (preferably metal, more preferably aluminium) having a metering valve disposed therein. Since the canister is preferably part of an MDI, the metering valve design is typically a function of providing a predetermined dosage or amount of the drug contained within the pressurised container to a user.

The valve typically comprises a valve body having an inlet port through which the pharmaceutical aerosol formulation may enter said valve body, an outlet port through which the pharmaceutical aerosol may exit the valve body and an open/close mechanism by means of which flow through said outlet port is controllable.

The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

The metering volumes are typically from 25 to 100 μl, such as 50 μl or 63 μl. Suitably, the valve body defines a metering chamber for metering an amount of medicament formulation and an open/close mechanism by means of which the flow through the inlet port to the metering chamber is controllable. Preferably, the valve body has a sampling chamber in communication with the metering chamber via a second inlet port, said inlet port being controllable by means of an open/close mechanism thereby regulating the flow of medicament formulation into the metering chamber.

The valve may be a metering valve in which the valve body has a metering chamber, a sampling chamber and therebetween a second sealing ring within which the stem is slidably movable, the valve stem having a transfer passage such that in the valve-closed position the dispensing passage is isolated from the metering chamber and the metering chamber is in communication with the sampling chamber via the transfer passage, and in the valve-open position the dispensing passage is in communication with the metering chamber and the transfer passage is isolated from the metering chamber.

The valve may also comprise a ‘free flow aerosol valve’ having a chamber and a valve stem extending into the chamber and movable relative to the chamber between dispensing and non-dispensing positions. The valve stem has a configuration and the chamber has an internal configuration such that a metered volume is defined therebetween and such that during movement between non-dispensing and dispensing positions the valve stem sequentially: (i) allows free flow of aerosol formulation into the chamber, (ii) defines a closed metered volume for pressurised aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurised aerosol formulation. A valve of this type is described in U.S. Pat. No. 5,772,085, the content of which is hereby incorporated herein by reference.

The valve may also have a structure and action similar to those aerosol valves described in European Patent Application No. EP-A-870,699 and PCT Patent Application No. WO99/36334, the content of each of which is hereby incorporated herein by reference.

The sealing ring and/or gasket may be formed by cutting a ring from a sheet of suitable material. Alternatively, the sealing ring and/or gasket may be formed by a moulding process such as an injection moulding, a compression moulding or a transfer moulding process.

Typically, the sealing ring and/or second sealing ring and/or gasket comprise an elastomeric material. The ring is typically resiliently deformable.

The elastomeric material may either comprise a thermoplastic elastomer (TPE) or a thermoset elastomer, which may optionally be cross-linked. The sealing ring and/or gasket may also comprise a thermoplastic elastomer blend or alloy in which an elastomeric material is dispersed in a thermoplastic matrix. The elastomers may optionally additionally contain conventional polymer additives. Such additives include but are not limited to processing aids, colorants, tackifiers, lubricants, silica, talc, or processing oils such as mineral oil in suitable amounts.

Suitable thermoset rubbers include butyl rubbers, chloro-butyl rubbers, bromo-butyl rubbers, nitrile rubbers, silicone rubbers, fluorosilicone rubbers, fluorocarbon rubbers, polysulphide rubbers, polypropylene oxide rubbers, isoprene rubbers, isoprene-isobutene rubbers, isobutylene rubbers or neoprene (polychloroprene) rubbers.

Suitable thermoplastic elastomers comprise a copolymer of about 80 to about 95 mole percent ethylene and a total of about 5 to about 20 mole percent of one or more comonomers selected from the group consisting of 1-butene, 1-hexene, and 1-octene as known in the art. Two or more such copolymers may be blended together to form a thermoplastic polymer blend.

Another suitable class of thermoplastic elastomers are the styrene-ethylene/butylene-styrene block copolymers. These copolymers may additionally comprise a polyolefin (e.g. polypropylene) and a siloxane.

Thermoplastic elastomeric material may also be selected from one or more of the following: polyester rubbers, polyurethane rubbers, ethylene vinyl acetate rubber, styrene butadiene rubber, copolyether ester TPE, olefinic TPE, polyester amide TPE and polyether amide TPE.

Other suitable elastomers include ethylene propylene diene rubber (EPDM). The EPDM may be present on its own or present as part of a thermoplastic elastomer blend or alloy, e.g. in the form of particles substantially uniformly dispersed in a continuous thermoplastic matrix (e.g. polypropylene or polyethylene). Commercially available thermoplastic elastomer blend and alloys include the SANTOPRENE™ elastomers. Other suitable thermoplastic elastomer blends include butyl-polyethylene (e.g. in a ratio ranging between about 2:3 and about 3:2) and butyl-polypropylene.

Typically, the sealing ring and/or the second sealing ring and/or gasket additionally comprises lubricant material. Suitably, the sealing ring and/or the second sealing ring and/or gasket comprises up to 30% by weight, preferably from 5 to 20% by weight, of lubricant material.

In addition, the stem may also comprise lubricant material. Suitably, the valve stem comprises up to 30%, preferably from 5 to 20% lubricant material by weight.

The term ‘lubricant’ herein means any material that reduces friction between the valve stem and seal. Suitable lubricants include silicone oil or a fluorocarbon polymer such as polytetrafluoroethane (PTFE) or fluoroethylene propylene (FEP).

Lubricant can be applied to the stem, stem gaskets or ferrule by any suitable process including coating and impregnation, such as by injection or by adding a reservoir of lubricant, which provides a constant supply of lubricant throughout the life of the product.

In medical use the canisters in accordance with the invention contain a pharmaceutical aerosol formulation comprising a medicament and a fluorocarbon or hydrogen-containing chlorofluorocarbon propellant.

Suitable propellants include, for example, C₁₋₄hydrogen-containing chlorofluorocarbons such as CH₂ClF, CClF₂CHClF, CF₃CHClF, CHF₂CClF₂, CHClFCHF₂, CF₃CH₂Cl and CClF₂CH₃; C₁₋₄hydrogen-containing fluorocarbons such as CHF₂CHF₂, CF₃CH₂F, CHF₂CH₃ and CF₃CHFCF₃; and perfluorocarbons such as CF₃CF₃ and CF₃CF₂CF₃.

Where mixtures of the fluorocarbons or hydrogen-containing chlorofluorocarbons are employed they may be mixtures of the above-identified compounds or mixtures, preferably binary mixtures, with other fluorocarbons or hydrogen-containing chloro-fluorocarbons for example CHClF₂, CH₂F₂ and CF₃CH₃. Preferably a single fluorocarbon or hydrogen-containing chlorofluorocarbon is employed as the propellant. Particularly preferred as propellants are C₁₋₄hydrogen-containing fluorocarbons such as 1,1,1,2-tetrafluoroethane (CF₃CH₂F) and 1,1,1,2,3,3,3-heptafluoro-n-propane (CF₃CHFCF₃) or mixtures thereof.

The pharmaceutical formulations for use in the canisters of the invention contain no components that provoke the degradation of stratospheric ozone. In particular the formulations are substantially free of chlorofluorocarbons such as CCl₃F, CCl₂F₂ and CF₃CCl₃.

The propellant may additionally contain a volatile adjuvant such as a saturated hydrocarbon for example propane, n-butane, isobutane, pentane and isopentane or a dialkyl ether for example dimethyl ether. In general, up to 50% w/w of the propellant may comprise a volatile hydrocarbon, for example 1 to 30% w/w. However, formulations which are free or substantially free of volatile adjuvants are preferred. In certain cases, it may be desirable to include appropriate amounts of water, which can be advantageous in modifying the dielectric properties of the propellant.

The invention is particularly useful with propellants (including propellant mixtures) which are more hygroscopic than P11, P114 and/or P12 such as HFA-134a and HFA-227.

A polar co-solvent such as C₂₋₆ aliphatic alcohols and polyols e.g. ethanol, isopropanol and propylene glycol, preferably ethanol, may be included in the drug formulation in the desired amount to improve the dispersion of the formulation, either as the only excipient or in addition to other excipients such as surfactants. Suitably, the drug formulation may contain 0.01 to 30% w/w based on the propellant of a polar co-solvent e.g. ethanol, preferably 0.1 to 20% w/w e.g. about 0.1 to 15% w/w. In aspects herein, the solvent is added in sufficient quantities to solubilise a part of, or all of, the medicament component, such formulations being commonly referred to as solution formulations.

A surfactant may also be employed in the aerosol formulation. Examples of conventional surfactants are disclosed in EP-A-372,777, the content of which is hereby incorporated herein by reference. The amount of surfactant employed is desirable in the range 0.0001% to 50% weight to weight ratio relative to the medicament, in particular, 0.05 to 5% weight to weight ratio.

The final aerosol formulation desirably contains 0.005-10% w/w, preferably 0.005 to 5% w/w, especially 0.01 to 1.0% w/w, of medicament relative to the total weight of the formulation.

Medicaments, which may be administered in the aerosol formulations, include any drug useful in inhalation therapy. The dispenser of the invention is in one aspect suitable for dispensing medicament for the treatment of respiratory disorders such as disorders of the lungs and bronchial tracts including asthma and chronic obstructive pulmonary disorder (COPD). In another aspect, the invention is suitable for dispensing medicament for the treatment of a condition requiring treatment by the systemic circulation of medicament, for example migraine, diabetes, pain relief e.g. inhaled morphine.

Accordingly, in one aspect, there is provided the use of a dispenser or MDI according to the invention for the treatment of a respiratory disorder, such as asthma and COPD. Alternatively, the present invention provides a method of treating a respiratory disorder such as, for example, asthma and COPD, which comprises administration by inhalation of an effective amount of an aerosol formulation as herein described from a dispenser or MDI of the present invention.

A further aspect of the invention provides the use of a dispenser or MDI according to the invention for the treatment of a condition requiring the systemic circulation of a medicament, such as, for example, migraine, diabetes, chronic pain. Alternatively, the present invention provides a method of treating a condition requiring the systemic circulation of medicament, such as, for example migraine, diabetes and chronic pain, which comprises administration by inhalation of an effective amount of an aerosol formulation as herein described from a dispenser or MDI or the present invention.

Appropriate medicaments may thus be selected from, for example, analgesics, e.g., codeine, dihydromorphine, ergotamine, fentanyl or morphine; anginal preparations, e.g., diltiazem; antiallergics, e.g., cromoglycate (e.g. as the sodium salt), ketotifen or nedocromil (e.g. as the sodium salt); antiinfectives e.g., cephalosporins, penicillins, streptomycin, sulphonamides, tetracyclines and pentamidine; antihistamines, e.g., methapyrilene; anti-inflammatories, e.g., beclomethasone (e.g. as the dipropionate ester), fluticasone (e.g. as the propionate or furoate ester), flunisolide, budesonide, rofleponide, mometasone (e.g. as the furoate ester), ciclesonide, triamcinolone (e.g. as the acetonide) or 6α,9α-difluoro-11β,-hydroxy-16α-methyl-3-oxo-17α-propionyloxy-androsta-1,4-diene-17β-carbothioic acid S-(2-oxo-tetrahydro-furan-3-yl)ester; antitussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol or 4-hydroxy-7-[2-[[2-[[3-(2-phenylethoxy)propyl]sulfonyl]ethyl]amino]ethyl-2(3H)-benzothiazolone; adenosine 2a agonists, e.g. 2R,3R,4S,5R)-2-[6-Amino-2-(1S-hydroxymethyl-2-phenyl-ethylamino)-purin-9-yl]-5-(2-ethyl-2H-tetrazol-5-yl)-tetrahydro-furan-3,4-diol (e.g. as maleate); α₄ integrin inhibitors e.g. (2S)-3-[4-({[4-(aminocarbonyl)-1-piperidinyl]carbonyl}oxy)phenyl]-2-[((2S)-4-methyl-2-{[2-(2-methylphenoxy)acetyl]amino}pentanoyl)amino]propanoic acid (e.g. as free acid or potassium salt), diuretics, e.g., amiloride; anticholinergics, e.g., ipratropium (e.g. as bromide), tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon; vaccines, diagnostics, and gene therapies. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g. hydrates) to optimise the activity and/or stability of the medicament.

Preferred medicaments are selected from albuterol, salmeterol, fluticasone propionate and beclomethasone dipropionate and salts or solvates thereof, e.g., the sulphate of albuterol and the xinafoate of salmeterol.

Medicaments can also be delivered in combinations. Preferred formulations containing combinations of active ingredients contain salbutamol (e.g., as the free base or the sulphate salt) or salmeterol (e.g., as the xinafoate salt) or formoterol (e.g. as the fumarate salt) in combination with an anti-inflammatory steroid such as a beclomethasone ester (e.g., the dipropionate) or a fluticasone ester (e.g., the propionate) or budesonide. A particularly preferred combination is a combination of fluticasone propionate and salmeterol, or a salt thereof (particularly the xinafoate salt). A further combination of particular interest is budesonide and formoterol (e.g. as the fumarate salt).

Particularly preferred formulations for use in the canisters of the present invention comprise a medicament and a C₁₋₄ hydrofluoroalkane particularly 1,1,1,2-tetrafluoroethane and 1,1,1,2,3,3,3-n-heptafluoropropane or a mixture thereof as propellant.

Conventional bulk manufacturing methods and machinery well known to those skilled in the art of pharmaceutical aerosol manufacture may be employed for the preparation of large scale batches for the commercial production of filled canisters. Thus, for example, in one bulk manufacturing method a metering valve is crimped onto an aluminium can to form an empty canister. The particulate medicament is added to a charge vessel and liquefied propellant is pressure filled through the charge vessel into a manufacturing vessel. The drug suspension is mixed before re-circulation to a filling machine and an aliquot of the drug suspension is then filled through the metering valve into the canister. Typically, in batches prepared for pharmaceutical use, each filled canister is check-weighed, coded with a batch number and packed into a tray for storage before release testing.

Each filled canister is conveniently fitted into a suitable channelling device prior to use to form a metered dose inhaler for administration of the medicament into the lungs or nasal cavity of a patient. Suitable channelling devices comprise for example a valve actuator and a cylindrical or cone-like passage through which medicament may be delivered from the filled canister via the metering valve to the nose or mouth of a patient e.g. a mouthpiece actuator. Metered dose inhalers are designed to deliver a fixed unit dosage of medicament per actuation or “puff”, for example in the range of 2 to 5000 microgram medicament per puff.

Administration of medicament may be indicated for the treatment of mild, moderate or severe acute or chronic symptoms or for prophylactic treatment. It will be appreciated that the precise dose administered will depend on the age and condition of the patient, the particular particulate medicament used and the frequency of administration and will ultimately be at the discretion of the attendant physician. When combinations of medicaments are employed the dose of each component of the combination will in general be that employed for each component when used alone. Typically, administration may be one or more times, for example from 1 to 8 times per day, giving for example 1,2,3 or 4 puffs each time. Each valve actuation, for example, may deliver 5 μg, 50 μg, 100 μg, 200 μg or 250 μg of a medicament. Typically, each filled canister for use in a metered dose inhaler contains 60, 100, 120 or 200 metered doses or puffs of medicament; the dosage of each medicament is either known or readily ascertainable by those skilled in the art.

For the avoidance of doubt, the use herein of the term “about” in reference to the value(s) of certain parameters is meant to include the exact value of that parameter, e.g. a reference to the relative amount of a material being “about Xg by weight” encompasses the relative amount being exactly Xg by weight.

1. A dispenser for dispensing a medicament comprising a canister for housing the medicament and a fluid propellant therefor and a drug-dispensing valve wherein one or more of the internal surfaces of the canister and/or valve comprises a fluorinated coating prepared from plasma polymerisation of one or more fluorinated monomers selected from the group consisting of CH₂FCF₃ and C₃F₆.

2. A dispenser according to claim 1, wherein the fluorinated coating is prepared from the plasma polymerisation of CH₂FCF₃.

3. A dispenser according to claim 1, wherein the fluorinated coating is prepared from the plasma polymerisation of C₃F₆.

4. A dispenser according to claim 1, wherein the fluorinated coating has a fluorine/carbon atomic ratio of greater than about 1.0.

5. A dispenser according to claim 4, wherein the fluorine/carbon atomic ratio is greater than about 1.2.

6. A dispenser according to claim 1, wherein the fluorinated coating comprises greater than about 10% CF₂ units.

7. A dispenser according to claim 1, wherein the fluorinated coating comprises greater than about 10% CF₂CF units.

8. A dispenser according to claim 1, wherein the fluorinated coating gives a contact angle of greater than about 80°.

9. A dispenser according to claim 8, wherein the fluorinated coating gives a contact angle of greater than about 90°.

10. A dispenser according to claim 1, wherein the fluorinated coating has a thickness in the range of about 1 to 200 nm.

11. A dispenser according to claim 10, wherein the thickness is in the range of about 10 to 100 nm.

12. A dispenser according to claim 1, wherein the fluorinated coating is provided on one or more internal surface of the canister.

13. A dispenser according to claim 1, wherein the fluorinated coating is provided on one or more internal surfaces of the valve.

14. A dispenser according to claim 13, wherein the fluorinated coating is provided on one or more internal surfaces of a metering chamber of the valve.

15. A dispenser according to claim 13, wherein the fluorinated coating is provided on a valve stem of the valve.

16. A dispenser according to claim 13, wherein the fluorinated coating is provided on one or more valve component parts selected from the group consisting of an upper stem seal, a lower stem seal, a neck gasket, a spring, a body and a ring.

17. A dispenser according to claim 1, wherein the fluorinated coating is prepared from the plasma co-polymerisation of one or more fluorinated monomers selected from the group consisting of CH₂FCF₃ and C₃F₆ and one or more additional non-fluorinated monomers.

18. A dispenser according to claim 17, wherein the one or more additional non-fluorinated monomers are selected from the group consisting of CH₄, C₂H₆, C₂H₄, N₂, O₂, H₂, C₃COO(C₆H₆)COOCH₃, HO(CH₂)₂OH, C₃H₃N and C₄H₆.

19. A drug-dispensing valve for use in a dispenser for dispensing a medicament in a fluid propellant, wherein one or more internal surface of said valve comprises a fluorinated coating prepared from plasma polymerisation of one or more fluorinated monomers selected from the group consisting of CH₂FCF₃ and C₃F₆.

20. A canister for housing a medicament in a fluid propellant, wherein one or more internal surface of said canister comprises a fluorinated coating prepared from plasma polymerisation of one or more fluorinated monomers selected from the group consisting of CH₂FCF₃ and C₃F₆.

21. A process for coating one or more internal surfaces of a canister and/or valve component with a fluorinated coating, said process comprising the steps of (i) placing the component to be coated in a chamber, (ii) evacuating the chamber, (iii) feeding a fluorinated monomer selected from the group consisting of CH₂FCF₃ and C₃F₆ into the chamber, (iv) applying sufficient power to generate a plasma, (v) igniting the plasma, (vi) extinguishing any unreacted plasma, and (vii) flushing the chamber.

22. A process according to claim 21, wherein one or more additional non-fluorinated monomers are fed into the chamber.

23. A process according to claim 22, wherein the ratio of fluorinated to non-fluorinated gas flow rate is continuously varied during the process.

24. A process according to claim 22, wherein the ratio of fluorinated to non-fluorinated gas flow rate is increased during the process.

25. A process according to claim 22, wherein the monomer gas is pure non-fluorinated monomer at the start of the process and pure fluorinated monomer gas at the end of the process.

26. A process according to claim 21, wherein the process is carried out at a power from 50 W to 450 W.

27. A process according to claim 21, wherein the process is carried out at a gas pressure of less than or equal to 70 mTorr.

28. A process according to claim 21, wherein the process is carried out at a gas flow rate from 50 cc/min to 200 cc/min.

29. A process according to claim 21, wherein the process is carried out at a tumbler speed from 1 to 15 rpm.

30. A process according to claim 21, wherein the process is carried out at a temperature from 20° C. to 100° C.

31. A process according to claim 21, wherein the plasma polymerisation process is carried out at a radio frequency in the range of 2 MHz to 200 MHz.

32. A process according to claim 31, wherein the radio frequency is approximately 13.56 MHz.

33. A process according to claim 21 in which the plasma is maintained for a duration of 30 minutes, or substantially 30 minutes.

34. A process according to claim 21, wherein said process comprises a pre-treatment step to remove surface contamination and/or activate the surface.

35. A process according to claim 34, wherein said pre-treatment step comprises plasma treatment of the components with oxygen or argon.

36. A dispenser according to claim 1 comprising a medicament in a fluid propellant under pressure, wherein the fluid propellant comprises a hydrofluoroalkane.

37. A method of preventing drug deposition in a dispenser for dispensing a medicament in a fluid propellant under pressure having a canister for housing the medicament and a drug-metering valve, the method comprising the use of a dispenser, a drug metering valve or a canister as claimed in claim 1.

38. (canceled)

39. A method of treating a respiratory disorder, which comprises administration by inhalation of an effective amount of a medicament in a fluid propellant from a dispenser as claimed in claim 1.

40. (canceled)

41. A method of treating a condition requiring systemic circulation of a medicament, which comprises administration by inhalation of an effective amount of a medicament in a fluid propellant from a dispenser as claimed in claim 1.
